(19) United States
(12) Patent Application Publication
(10) Pub. No.: US 2022/0157193 A1
(43) Pub. Date: May 19, 2022

(54) MECHANISMS AUTHORING TOOL AND DATA COLLECTION SYSTEM

(71) Applicants: Julia English WINTER, Bloomfield Hills, MI (US); Joseph ENGALAN, Royal Oak, MI (US)

(72) Inventors: Julia English WINTER, Bloomfield Hills, MI (US); Joseph ENGALAN, Royal Oak, MI (US)

(21) Appl. No.: 17/435,647

(22) PCT Filed: Mar. 2, 2020
(86) PCT No.: PCT/US2020/020612

§ 371 Date: Sep. 1, 2021

Related U.S. Application Data
(60) Provisional application No. 62/812,415, filed on Mar. 1, 2019.

(51) Int. Cl. G09B 23/24; G09B 23/26; G16C 20/80; G16C 20/20

(52) U.S. Cl. CPC G09B 23/24; G16C 20/20; G16C 20/80; G09B 23/26

(57) ABSTRACT

A method for authoring and using a chemical mechanism includes a step of authoring a chemical mechanism problem to be solved by a user with an authoring tool. The chemical mechanism problem presents a user with chemical renderings of starting chemical compounds to be rearranged in a predetermined series of steps to form a predetermined final chemical compound. The authoring tool being implemented by an authoring computer device having a processor and a display. The chemical renderings of the starting chemical compounds are intended to be displayed on a user computer device. Therefore, the steps of the chemical problem created by the author recorded to a suitable storage medium. A series of inputs from the user are received on a user tool on the user computer device for moving atoms and or bonds in the chemical rendering of the starting chemical compounds to reproduce a chemical mechanism.

### CROSS-REFERENCE TO RELATED APPLICATIONS

This application claims the benefit of U.S. provisional application Ser. No. 62/812,415 filed Mar. 1, 2019, the disclosure of which is hereby incorporated in its entirety by reference herein.

### TECHNICAL FIELD

In at least one aspect, the present invention is related to computer processor applications for teaching chemical reaction mechanisms.

### BACKGROUND

The widespread use of computers and smart devices has significantly changed the manner in which people play, learn and study. Video games are perhaps the earliest form of electronic device-based application that has attained general acceptance. More recently, electronic books are becoming more and more common and are expected to surpass paper books in the near future. Similarly, online education has become an accepted alternative to classroom study.

For the most part, video games, though widespread, provide little educational benefit. The typical video game provides significant visual stimulation and perception of action. Educational video games do exist but tend to be directed more to the elementary school level. Advanced electronic games such as electronic crossword puzzles are typically just direct conversions of the paper game to electronic form. Few electronic games target an older audience to teach advanced scientific and engineering topics.

Accordingly, there is a need for advanced computer games that are enjoyable for users while teaching difficult scientific and engineering concepts.

### SUMMARY

In at least one aspect, a mechanisms authoring and data collection system is provided. The mechanisms authoring and data collection system implements a computer implemented method a step of authoring a chemical mechanism problem to be solved by a user with an authoring tool, the chemical mechanism problem presenting the user with chemical renderings of starting chemical compounds to be rearranged in a predetermined series of steps to form a predetermined final chemical compound. The authoring tool is implemented by an authoring computer device having a processor and a display. The chemical renderings of the starting chemical compounds can be displayed on a user computer device. A series of inputs from the user on a user tool are received on the user computer device for moving atoms and or bonds in the chemical rendering of the starting chemical compounds to reproduce a chemical mechanism. A monitoring tool characterizes and stores moves made by the users. The user moves and characterizations thereof are stored on a centralized computer device.

In another aspect, an authoring tool allows designers to create a mechanism problem (e.g., a mechanism puzzle) and designate which both which bonds to make and break as well as the order in which the bonds can be manipulated. By allowing the designer the freedom to choose the method by which the puzzle is solved, there is no need to build in additional chemical algorithms, beyond structure recognition, geometric layout, and formal charge calculations.

In still another aspect, a user tool is provided. The user tool is implemented on a user computer device. The user present a chemical mechanism problem to one or more users. The user tool receives one or more inputs from the user for moving atoms and or bonds in the chemical rendering of the starting chemical compounds to reproduce a chemical mechanism as a solution to the chemical mechanism problem.

In still another aspect, a monitoring tool is provided. The monitoring tool is implemented on a monitor computer device. The monitoring tool the tracking of one or a plurality of user's moves in attempting to solving a mechanism problem. The monitoring tool may also store the users' moves on a centralized computer device.

In still another aspect, a data collection system is provided that collects the moves of a plurality of users attempting to solve a mechanism problem. The system classifies moves as either correct or incorrect moves.

In still another aspect, user moves in solving a mechanism problem are coded as to type, (such as nucleophilic attack or deprotonation).

In still another aspect, user moves are coded as error moves, which correct themselves when a user performs the move. Moves that are not labeled (e.g., that is events that automatically correct themselves) are stored in the database.

In still another aspect, a mechanisms authoring and data collection system present an author with a first listing of atoms is presented by an authoring tool to be used in authoring a chemical mechanic problem. Characteristically, the author selects an atom from the first listing of atoms and placing the selected atom on a design region.

In still another aspect, a mechanisms authoring and data collection system displays implied hydrogen atoms as a letter “H” orbiting a carbon atom represented by a letter “C” on either an authoring computer device or a user computer device. Characteristically the number of letters “H” represents the number of implied hydrogen atoms bonded to the carbon atom. Typically, implied hydrogen atoms orbit on displayed circles surrounding the letter “C.”

In yet another aspect, a mechanisms authoring and data collection system identifies frequent errors made by individual users.

In yet another aspect, a mechanisms authoring and data collection system identifies frequent errors made by a plurality of users.

In yet another aspect, a mechanisms authoring and data collection system identifies patterns of steps made by individual users.

In yet another aspect, a mechanisms authoring and data collection system identifies patterns of steps made by a plurality of users.

In yet another aspect, a mechanisms authoring and data collection system identify potential errors that the user may commit are identified during authoring of the chemical mechanism problem.

In yet another aspect, a mechanisms authoring and data collection system display a message to be displayed if a user commits an identified potential error.

### BRIEF DESCRIPTION OF THE DRAWINGS

FIG. 1A provides a schematic of a user interface for an authoring tool for creating a mechanism problem showing atoms that can be used to create a rendering of the reactive atoms and molecules that participate in a mechanism problem.

FIG. 1B provide a schematic of a data entry box for the authoring tool for creating a mechanism problem.

FIG. 1C provide a schematic of a data entry box for the authoring tool for creating a mechanism problem.

FIGS. 2A, 2B, 2C, and 2D provide exemplary graphical users interfaces for authoring a mechanism problem.

FIGS. 3A, 3B, and 3C provide exemplary graphical users interfaces for a user attempting to solve a mechanism problem.

FIG. 4 provides a screenshot showing the rendering of implied hydrogen atoms.

FIGS. 5A and 5B depict user interfaces that provide a monitor (e.g., a teacher) with feedback regarding users' progress.

FIGS. 6A, 6B, 6C, and 6D are schematics illustrating the tracking of one or a plurality of user's moves in attempting to solving a mechanism problem.

FIG. 7 demonstrates the attempts from a plurality of users can be tracked and the results stored.

FIGS. 8A and 8B provides schematics demonstrating a method for evaluating (e.g., scoring or grading) users' attempts in solving the mechanism puzzle.

FIGS. 9A, 9B, and 9C depict application of a grading rubric to a multistep addition reaction mechanism.

FIG. 10 provides a schematic showing that errors that users make can aid in identifying concepts that present difficulties.

FIG. 11 provides a schematic of a networked chemical mechanism evaluation and data collection system is provided.

FIG. 12 provides a schematic of a computer devices used to implement the methods of the invention.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can betaken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

The term “connected to” means that the electrical components referred to as connected to are in electrical communication. In a refinement, “connected to” means that the electrical components referred to as connected to are directly wired to each other. In another refinement, “connected to” means that the electrical components communicate wirelessly or by a combination of wired and wirelessly connected components. In another refinement, “connected to” means that one or more additional electrical components are interposed between the electrical components referred to as connected to with an electrical signal from an originating component being processed (e.g., filtered, amplified, modulated, rectified, attenuated, summed, subtracted, etc.) before being received to the component connected thereto.

The term “electrical communication” means that an electrical signal is either directly or indirectly sent from an originating electronic device to a receiving electrical device. Indirect electrical communication can involve processing of the electrical signal, including but not limited to, filtering of the signal, amplification of the signal, rectification of the signal, modulation of the signal, attenuation of the signal, adding of the signal with another signal, subtracting the signal from another signal, subtracting another signal from the signal, and the like. Electrical communication can be accomplished with wired components, wirelessly connected components, or a combination thereof.

The term “electronic component” refers is any physical entity in an electronic device or system used to affect electron states, electron flow, or the electric fields associated with the electrons. Examples of electronic components include, but are not limited to, capacitors, inductors, resistors, thyristors, diodes, transistors, etc. Electronic components can be passive or active.

The term “electronic device” or “system” refers to a physical entity formed from one or more electronic components to perform a predetermined function on an electrical signal.

It should be appreciated that in any figures for electronic devices, a series of electronic components connected by lines or arrow (e.g., wires or buses) indicates that such electronic components are in electrical communication with each other. Moreover, when lines directed connect one electronic component to another, these electronic components can be connected to each other as defined above.

The term “server” refers to any computer, computer device, mobile phone, desktop computer, notebook computer or laptop computer, distributed system, blade, gateway, switch, processing device, or combination thereof adapted to perform the methods and functions set forth herein.

The term “tool” refers to an executing program on a computer device that provides at least one functionality of the present invention set forth below.

The term “computer device” refers generally to any device that can perform at least one function, including communicating with another computer device. Examples of computer devices include bust are not limited to, smartphones, laptop computers, desktop computers, tablets (e.g., iPad), servers, and the like. Sometimes a computer device is referred to as a computer. Sometimes, a computer device is referred to as a computing device.

The term “graphical control element” means an element of interaction, such as a button or a scroll bar, that is capable of being manipulated by a user for purposes of entering commands or causing some associated action in a computer device that presents or contains the element.

The term “one or more” means “at least one” and the term “at least one” means “one or more.” The terms “one or more” and “at least one” include “plurality” as a subset.

When a computer device is described as performing an action or method step, it is understood that the computer devices is operable to perform the action or method step typically by executing one or more line of source code. The actions or method steps can be encoded onto non-transitory memory (e.g., hard drives, optical drive, flash drives, and the like).

Throughout this application, where publications are referenced, the disclosures of these publications in their entireties are hereby incorporated by reference into this application to more fully describe the state of the art to which this invention pertains.

In general, a mechanisms authoring and data collection system includes an authoring tool, a user tool (e.g., used by a student), and optionally a monitoring tool (e.g., used by a teacher) as explained below in more detail. In one variation, the authoring tool, the user tool, and the monitoring tool can be individually provided. In another variation, the authoring tool and the monitoring tool are provided as part of a single software package. In still another variation, the authoring tool, the user tool, and the monitoring tool are provided as part of a single software package.

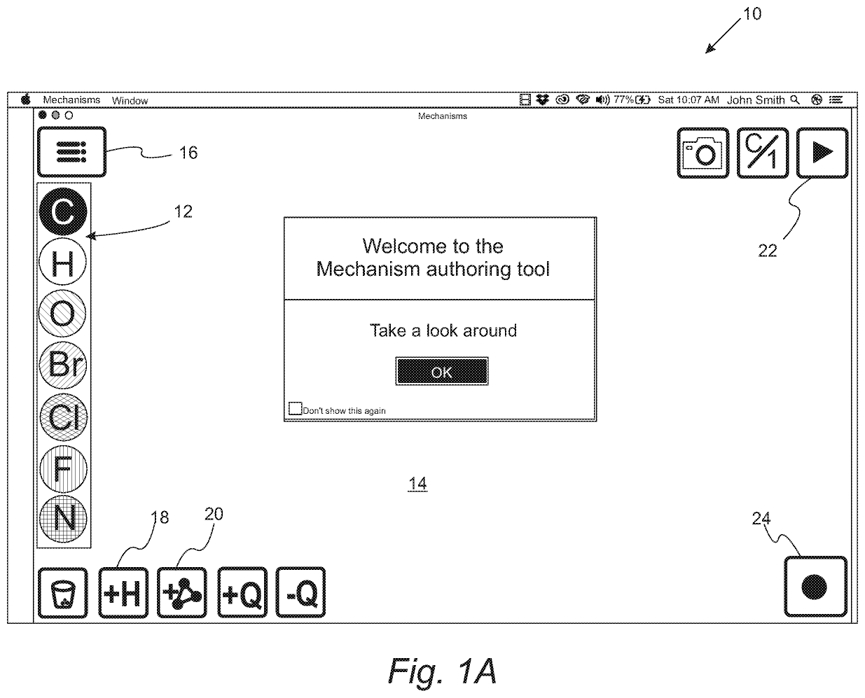

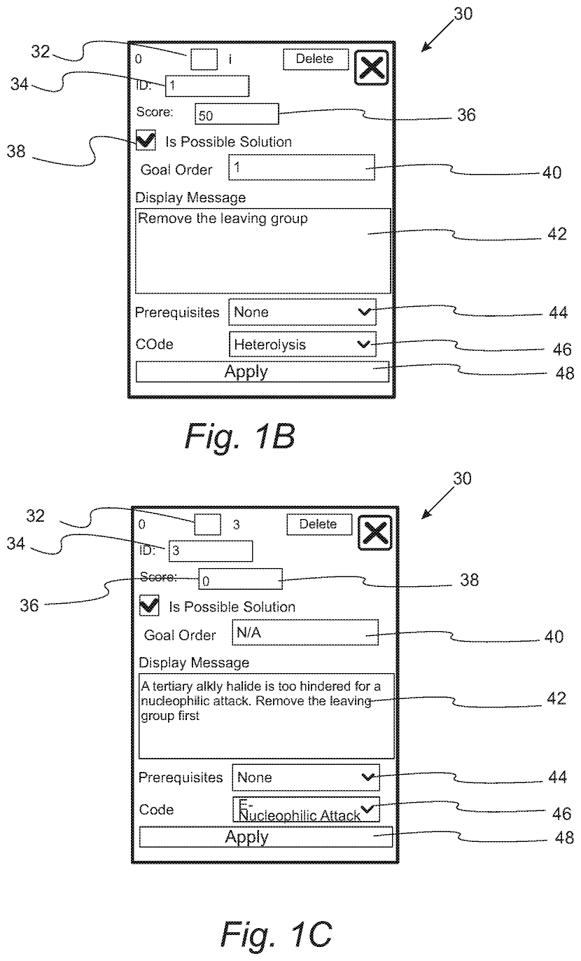

With reference to FIGS. 1A, 1B, and 1C, a method for authoring (i.e., an authoring and using a chemical mechanism problem is provided. Typically, the method is implemented on a computer device having a computer processor that executes or assists in executing the steps of the method. The authoring tool being implemented by an authoring computer device having a processor and a display. The method includes a step of authoring a chemical mechanism problem to be solved by a user with an authoring tool. As depicted in FIG. 1A, graphical user interface 10 presents an author with a listing 12 of atoms to be used in creating atoms and molecules that are involved in a chemical mechanism to be rendered on a display. The author selects atoms from listing 12 that are displayed in display region 14 of window 10. Graphical control element 16 allows the author to save or load chemical mechanism problems. Graphical control element 18 allows the author to add hydrogen atoms to display region 14 while control element 20 allows the author to add rendering of molecules or chemical moieties (e.g., H₂O, acetic acid, ethanol, hydroxide, etc.) to display region 14. Graphical control element 22 initiates testing of (i.e., running) a chemical mechanism problem created by the authoring tool while graphical control element 24 initiates recording the actions taken by a user when testing the chemical mechanism problem.

In a refinement, the author associates a code for each move in the mechanism problem is coded as to type, such as nucleophilic attack or deprotonation by entering such data in an input data entry box. In addition, moves are coded as error moves, which correct themselves when a user performs the move. For example, on the device, the bond automatically re-forms (in the case of bond-breaking) or breaks apart (in the case of bond-making). When this happens on the device the two atoms that are part of the error move have a jagged edge and a sound plays to indicate that the bond breaking or making was in error. FIG. 1B illustrates a data entry box for a single correct or preferred step of the mechanism problem while FIG. 1C illustrates a data entry box for a single incorrect or not preferred step of the mechanism problem. In each of these cases, the author enters data into data entry box 30 which includes field 34 for entering an ID for the data, field 36 for entering a score to be awarded is the user makes the associated step, field 38 for entering an indication that the associated step is a part of a possible solution to the mechanism problem, field 40 for entering a goal order that indicates the position of the associated step in the mechanism problem, field 42 for entering a display message for the associated step, field 44 for entering prerequisites for the associated step, and field 46 for entering the code for the associated step. In this context, the associated step is the single step that a user may attempt associated with the data being entered by the author. Entered data is stored in a database. A prerequisite is a move that must be made before another can take place. Some states are achievable performing valid moves. However, in order to test whether a user understands a step, they must first reach a prerequisite state. The points for a state are only awarded if they first hit the prerequisite state. Otherwise, the state is treated as just a regular valid state. For example, 1) A nucleophile attacks a carbon of a carbonyl (makes a bond with from the electron pair to the carbon) 2) the pi bond of the C═O must break and transfer electrons to the Oxygen resulting in a tetrahedral carbon, 3) The leaving group attached the carbon in the carbonyl can only depart after step 2 has been completed. In another example, this method in authoring to prompt students to show all resonance forms in a reaction. In the case of Electrophilic Aromatic Substitution, users must 1) attach the electrophile with a pi bond from the aromatic ring 2+) show the resonance stabilization by moving pi electrons around the ring 3) only after the steps 2+ can the ring be deprotonated to re-form the aromatic ring and substitute for the proton. Steps 2+ are the prerequisites for step 3.

In a refinement, moves that are not labeled can events that automatically correct themselves, are stored in the database. Invalid moves are recorded so they can be further analyzed by the system to determine which concepts the user has a solid understanding of and which concepts the user doesn't fully understand. This will enable the system to further help the user by allowing the system to report misunderstood concepts to the user or the assessor. Table 1 provides an example of a coding strategy. In a refinement, the steps of the chemical problem created by the author recorded to a suitable storage medium (e.g., ROM, hard drive, etc.)

TABLE 1
List of moves that are labeled in a mechanism problem

| Label | Description |
|---|---|
| B | BaseAttack |
| E | Deprotonation |
| N | NueclophilicAttack |
| A | Alkyl Shift |
| H | HydrideShift |
| R | Resonance |
| T | PiBondAttack |
| M | PiBondMake |
| K | PiBondBreak |
| Y | Heterolysis |
| O | Protonation |
| b | E-BaseAttack |
| e | E-Deprotonation |
| n | E-NueclophilicAttack |
| a | E-AlkylShift |
| h | E-HydrideShift |
| r | E-Resonance |
| t | E-PiBondAttack |
| m | E-PiBondMake |
| k | E-PiBondBreak |
| y | E-Heterolysis |
| o | E-Protonation |

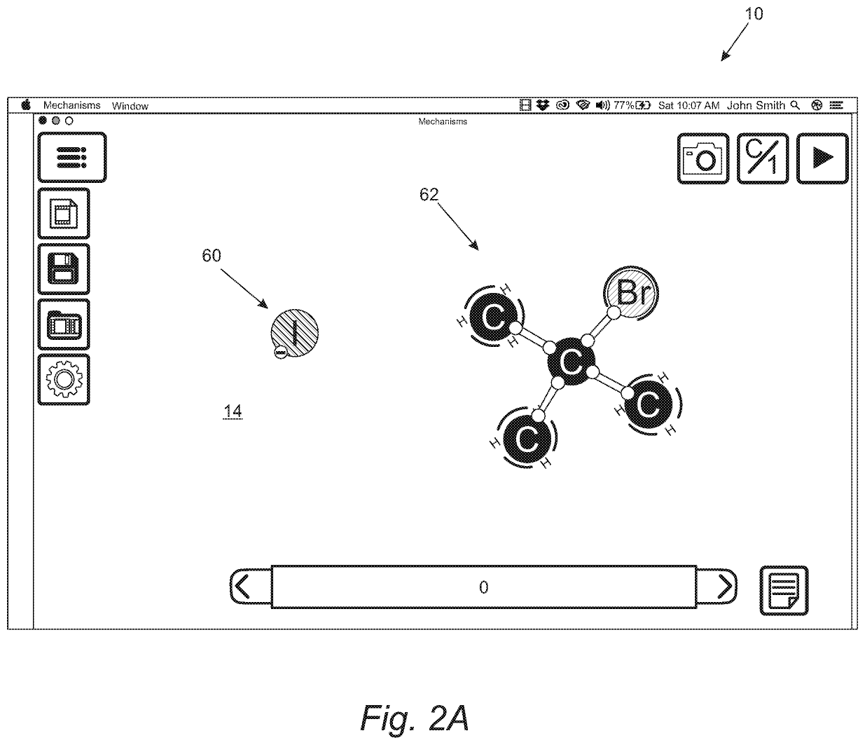

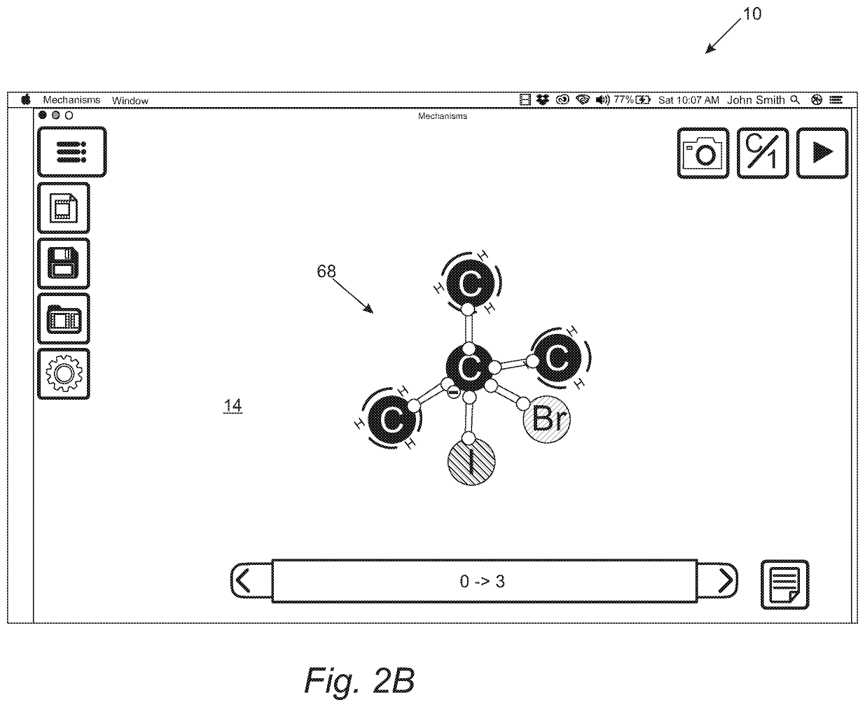

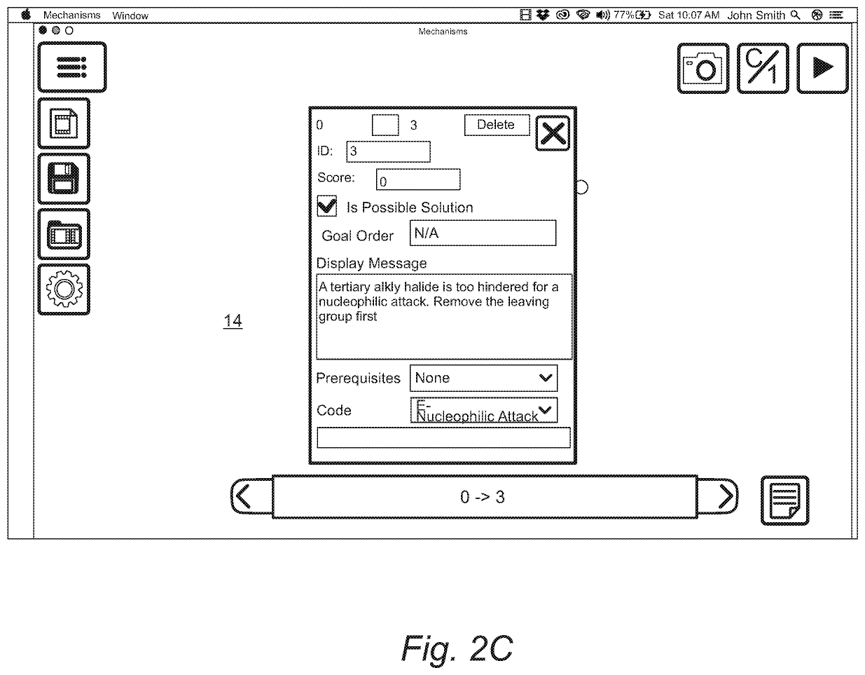

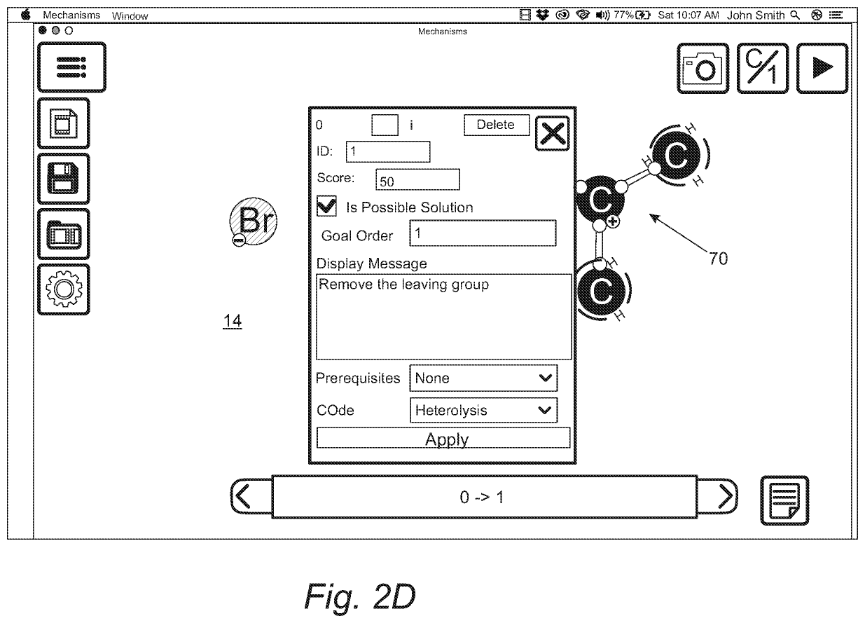

FIGS. 2A, 2B, 2C, and 2D illustrate the authoring of a mechanism problem. The particular example depicted in an S_{N}1 mechanism problem. For example, as depicted in FIG. 2A, uses interface 10 of FIG. 1A to create chemical renderings 60, 62 of starting chemical compounds to be rearranged by the user in a predetermined series of steps to form a predetermined final chemical compound. In a refinement, chemical renderings are stored using the simplified molecular-input line-entry system (SMILES) is a specification in the form of a line notation for describing the structure of chemical species using short ASCII strings.

In a refinement, implied hydrogen atoms for carbon atoms rendered in display region 14 are displayed by a letter “H” orbiting a carbon atom represented by a letter “C” on either the authoring computer device or the user computer device where the number of letters “H” representing the number of implied hydrogen atoms bonded to the carbon atom. In FIG. 2B, the author manipulates the rendering of the starting chemical compounds to form a potential reaction product 66 is an incorrect step. The renderings are manipulated by the author using a pointing device (e.g., a mouse or a finger for a touch screen) to move atoms and electrons (e.g., moving bonds). In FIG. 2C the author enters the data associated with the incorrect step depicted in FIG. 2B. FIG. 2D illustrates data entry for a correct step in which reactive product 70 is formed.

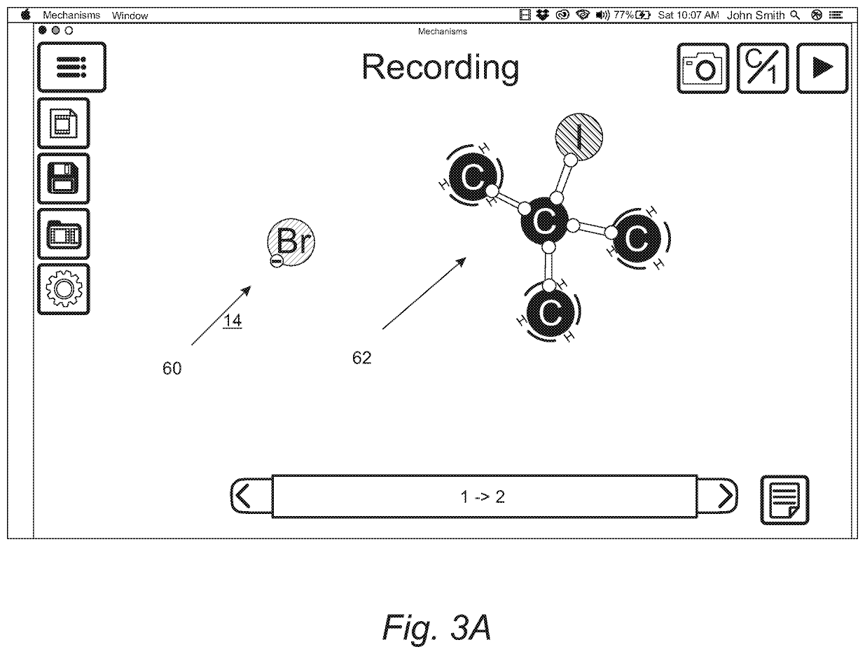

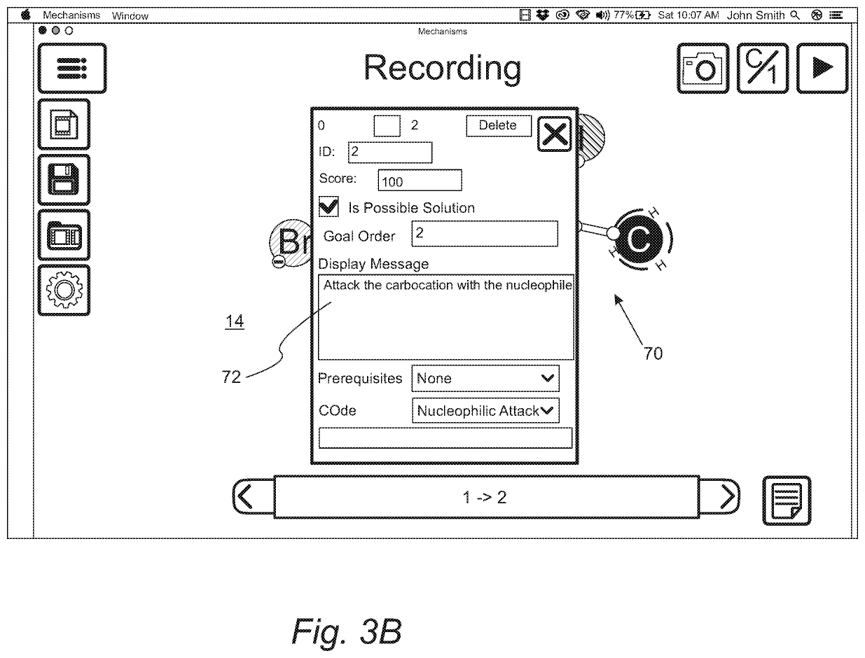

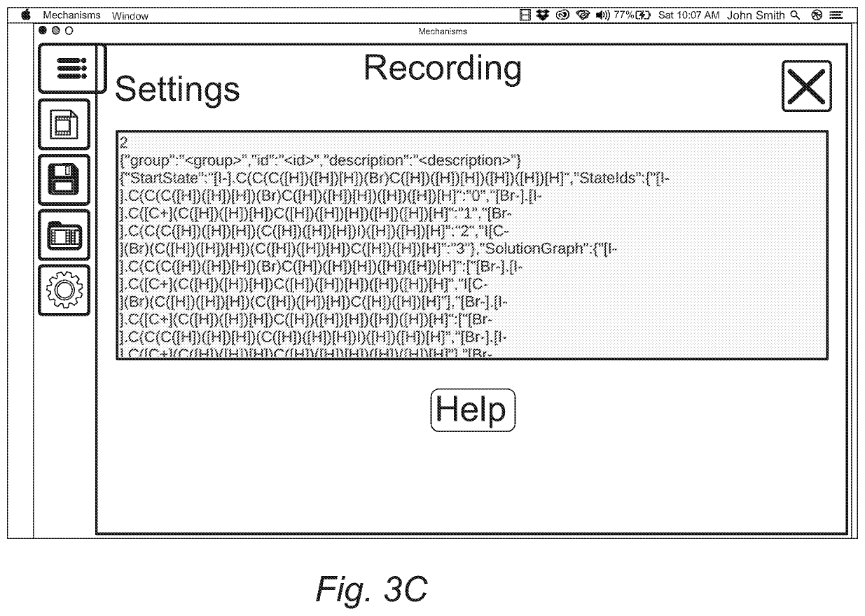

FIGS. 3A, 3B, and 3C illustrate a user attempting to solve a mechanism problem. The chemical renderings of the starting chemical compounds are intended to be displayed on a user computer device using a user tool (i.e., an executing program on the user computer device). FIG. 3A presents chemical renderings 60, 62 of starting chemical compounds to be rearranged by the user typically using a pointing device (e.g., a mouse or a finger when the user computer device includes a touch screen). In this rearrangement, atoms and electrons may be moved by the user manipulating the chemical renderings of the starting chemical compounds. Characteristically, the user tool replaces curved arrows used in paper construction of chemical mechanism with the direct movement of electrons. For example, the user selects a chemical bond attached to a first source atom and second atom. The user drags the bond off of the second atom and onto a target atom to form a bond thereto. Similarly, the user can drag an electron(s) from a lone pair on a first atom onto a target atom for forming a bond thereto. In a refinement, the user program automatically adjusts the electric charge on atoms after a user rearrangement. In another variation, the user provides the electric charges for the rearranged atoms. The user can perform multiple steps in a multi-step mechanism. A series of inputs from the user are received on a user tool on the user computer device for moving atoms and or bonds in the chemical rendering of the starting chemical compounds to reproduce a chemical mechanism. FIG. 3B depicts a situation where a user has correctly manipulated the reactant rendering to form the first mechanism product 70. The user is then optionally provided a hint 72 for the next step. FIG. 3A shows the string sequences for all of the molecular structures formed while the user is attempting to solve the mechanism problem. Moves made by the users are characterized and stored. In a refinement, the moves are collected on a centralized computer device (e.g., a centralized server). In a refinement, the user tool can guide a user through the mechanism problem with goals. Mover, the user tool can provide instant feedback and hints (e.g., the description added during creation of the mechanism problem, see FIGS. 1B and 1C). Typically, every move by the user is recorded and evaluated. Advantageously, the user tool can show a user's progress. In a refinement, the user tool can show mechanism problems attempted, and/or if assignments were late.

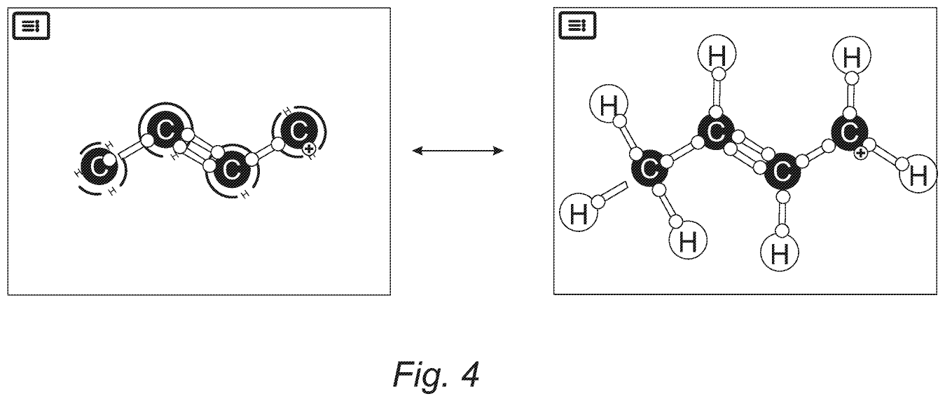

With reference to FIG. 4, implied hydrogen atoms are displayed by a letter “H” orbiting a carbon atom represented by a letter “C” on either the authoring computer device or the user computer device. The number of letters “H” representing the number of implied hydrogen atoms bonded to the carbon atom. Implied hydrogen atoms orbit on displayed circles surrounding the letter “C.” In a refinement, the size of the letter “H” is smaller than the size of the letter “C.” around which the “H's” orbit. Characteristically, upon actuation, each letter “H” is replaced by a rendering of a hydrogen atom bonded to carbon.

The method set forth herein enhances chemical education in a number of ways. In this regard, frequent errors made by individual users can be identified. Similarly, frequent errors made by a plurality of users can be identified. The data collection allows identifying patterns of steps made by individual users or a plurality of users to be identified. Such patterns can be identified with an expert system (e.g., modeling expert analysis) or by a trained neural network.

Potential errors that the user may commit are identified during the authoring of the chemical mechanism problem. As depicted in FIG. 2C, the author can create a message to be displayed if a user commits an identified potential error.

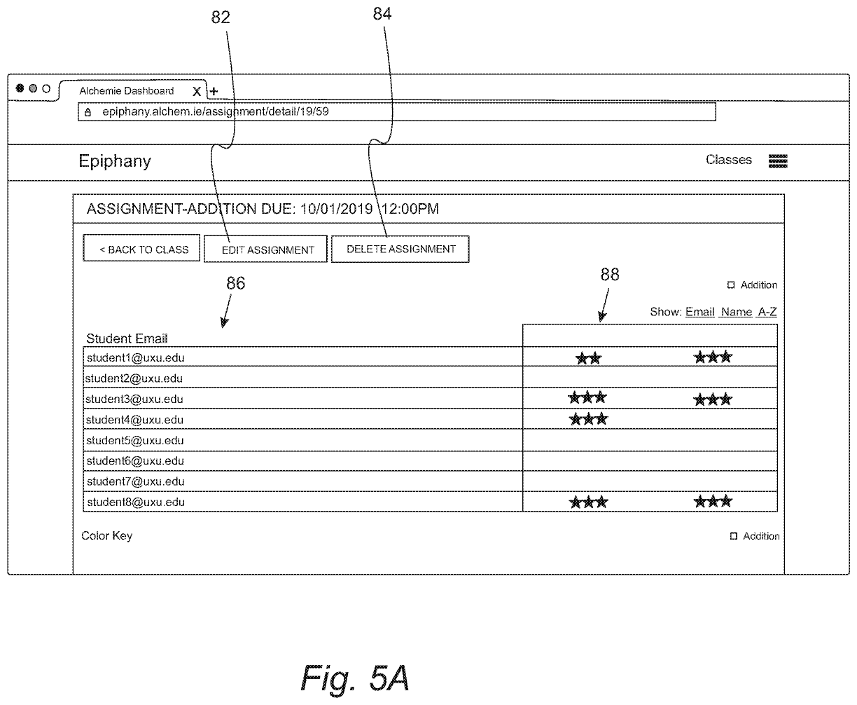

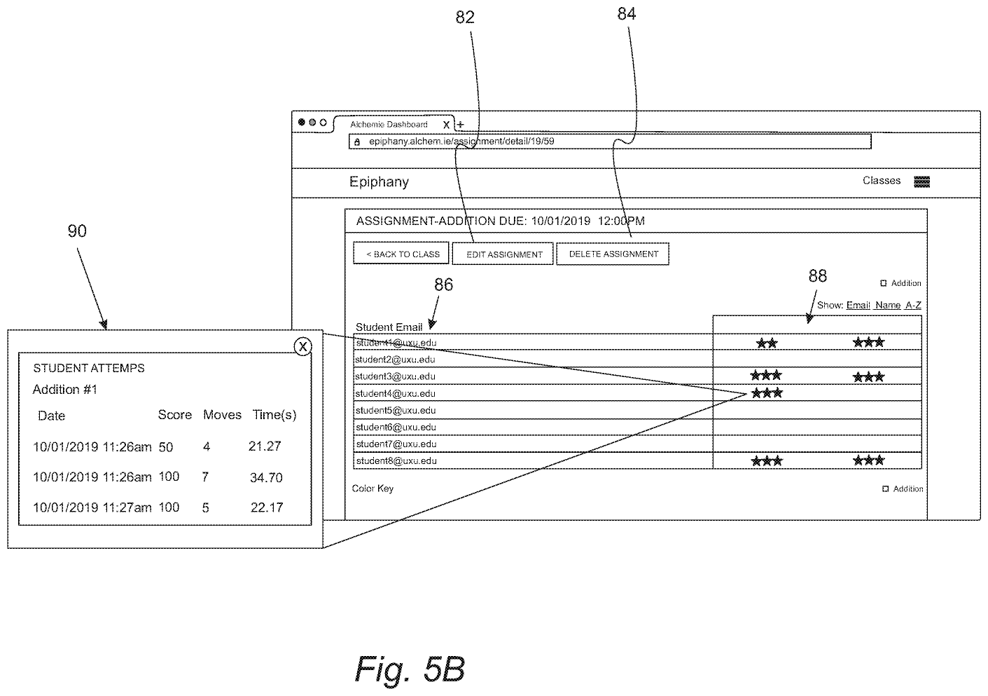

FIGS. 5A and 5B depict user interfaces that provide a monitor (e.g., a teacher) with feedback (i.e., the monitor feature) regarding users' progress. Graphical interface 80 provides such feedback to the monitor. In a refinement, the monitor feature is part of a monitoring tool described below in more detail. The monitor can edit assignments (e.g., functionality initiated with control element 82) and delete assignments (e.g., functionality initiated with control element 84) with this interface. Students are identified in student ID fields 86 with progress indicated in progress fields 88. FIG. 5B shows that additional progress information 90 can be obtained for each user. For example, the monitor can actuate the student identifier and/or the related progress field and/or another control element to display additional progress information 90.

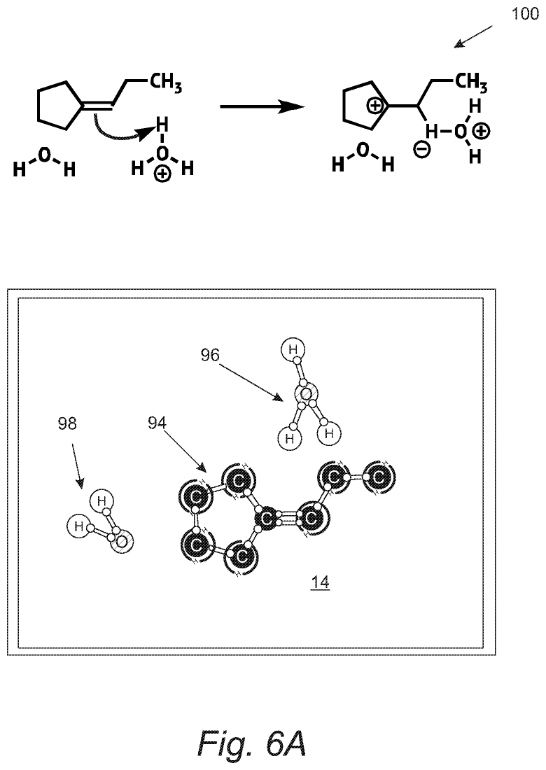

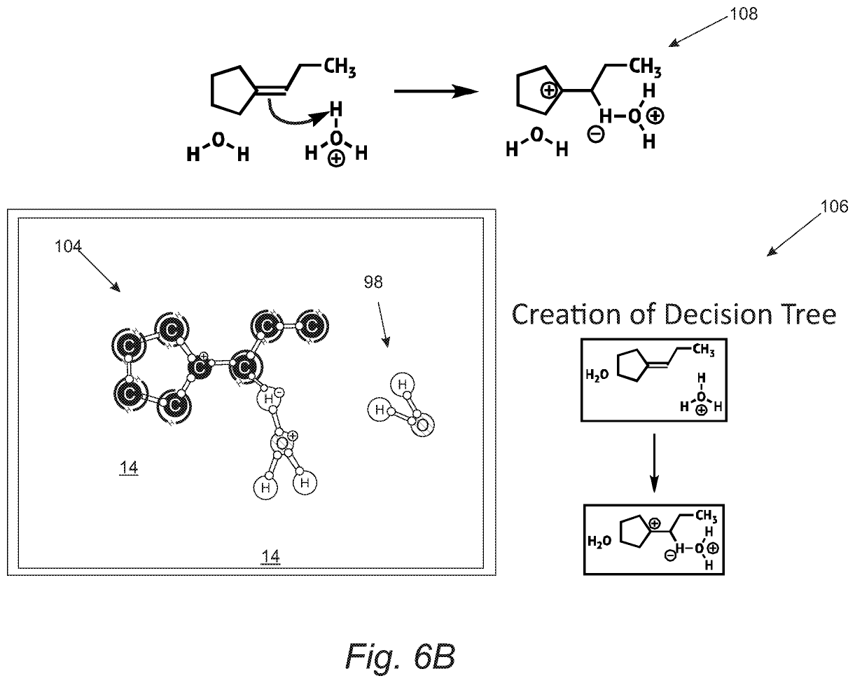

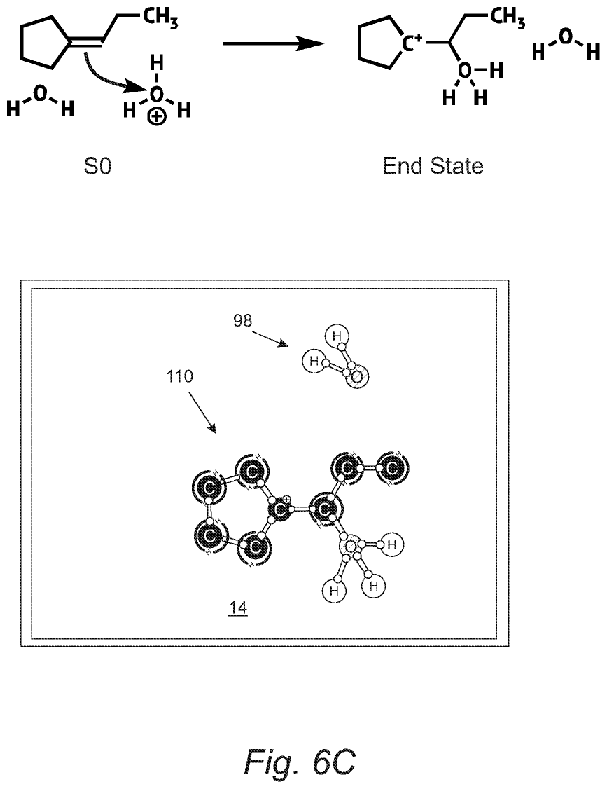

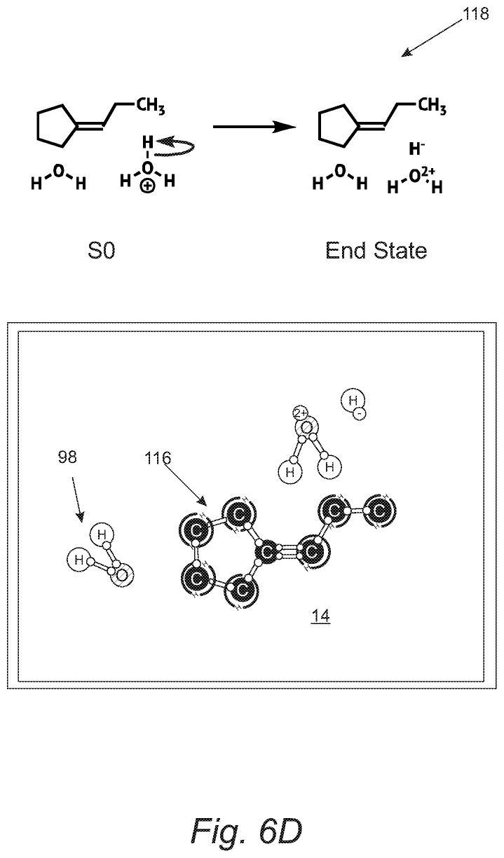

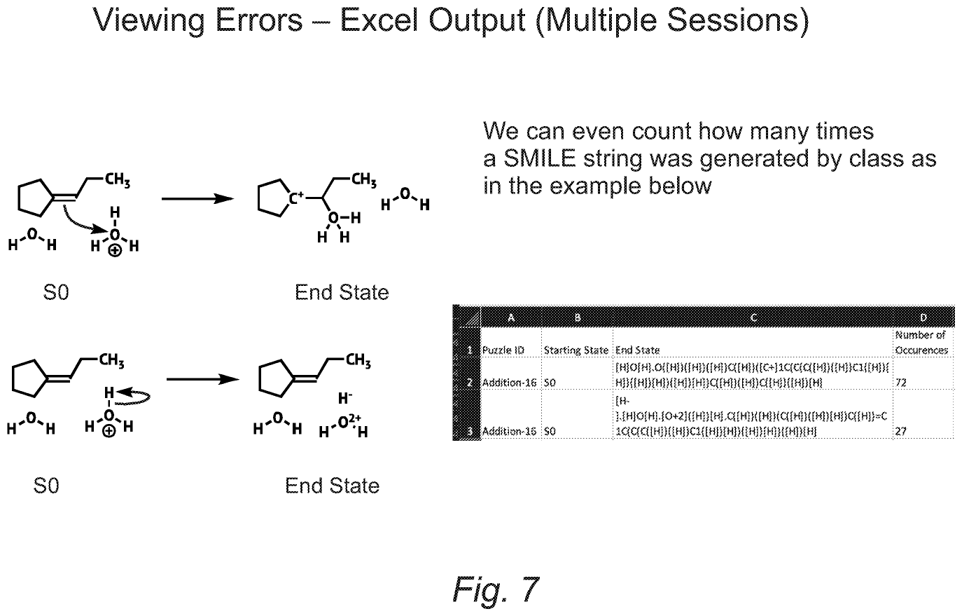

FIGS. 6A, 6B, 6C, and 6D provide a schematic illustrating the tracking of one or a plurality of user's moves in attempting to solving a mechanism problem. In a refinement, a monitoring tool can be used for tracking. In this regard, a string molecule encoding such as SMILE strings can be used to monitor which structures were formed while a user was attempting to solve the mechanism problem. For example, FIG. 6A depicts a mechanism problem for an addition reaction. For this problem, chemical renderings 94, 96, 98 of starting chemical compounds are presented in display region 14. FIG. 6A also provides the chemical mechanism 100 for this step draw in standard form with an arrow indicating electron movement. FIG. 6B depicts the result of a user correctly moving electrons in chemical renderings 94, 96, 98 of starting chemical compounds to form a rendering of a first correct intermediate compound 104. This user action can then be used to create part 106 of a decision tree. FIG. 6B also provides the chemical mechanism 108 for this step draw in standard form with an arrow indicating electron movement. This user action can then be used to create part of a decision tree. FIG. 6C depict depicts the result of a user incorrectly moving electrons in chemical renderings 94, 96, 98 of starting chemical compounds to form a rendering of first incorrect intermediate compound 116. FIG. 6C also provides the chemical mechanism 118 for this step draw in standard form with an arrow indicating electron movement. This user action can then be used to create part of a decision tree. FIG. 7 demonstrates the attempts from a plurality of users can be tracked and the results stored. For example, the monitor tool can count how the number of times a particular molecular structure was generated.

In a variation, the monitoring tool tracks one or a plurality of user's moves in attempting to solving a mechanism problem. The present invention is not limited by the number of users that can be tracked. Therefore, the system can track 1 to 10,000 or more users. The monitoring tool is operable to identify frequent errors made by individual users or by a plurality of users. In another refinement, the monitoring tool identifies patterns of steps made by individual users or by a plurality of users. In yet another refinement, the monitoring tool identifies potential errors that the user may commit are identified during the authoring of the chemical mechanism problem.

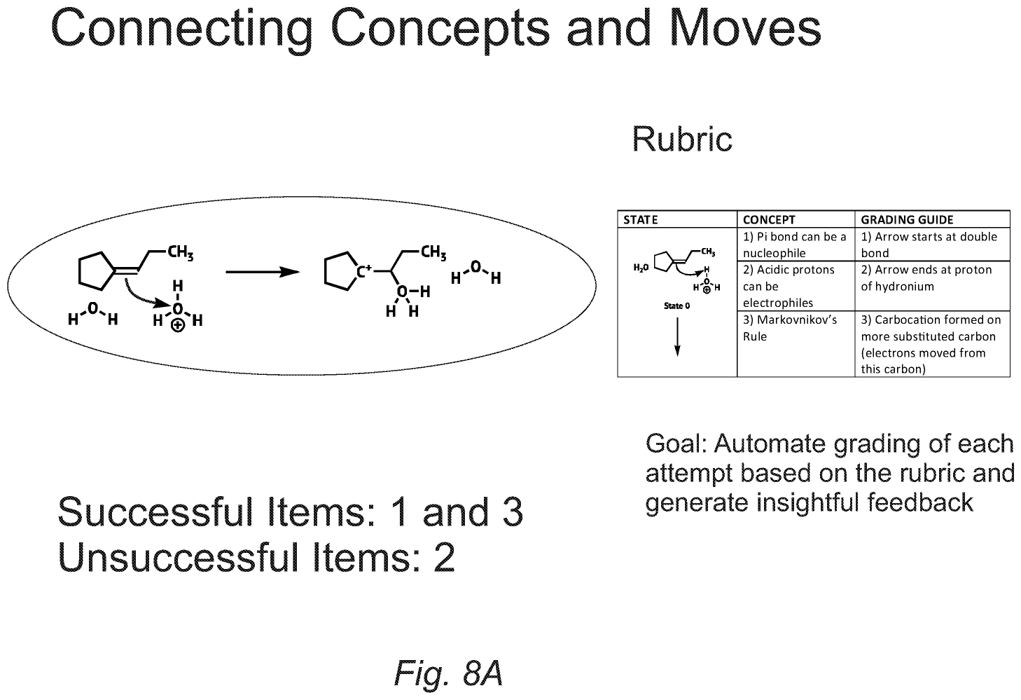

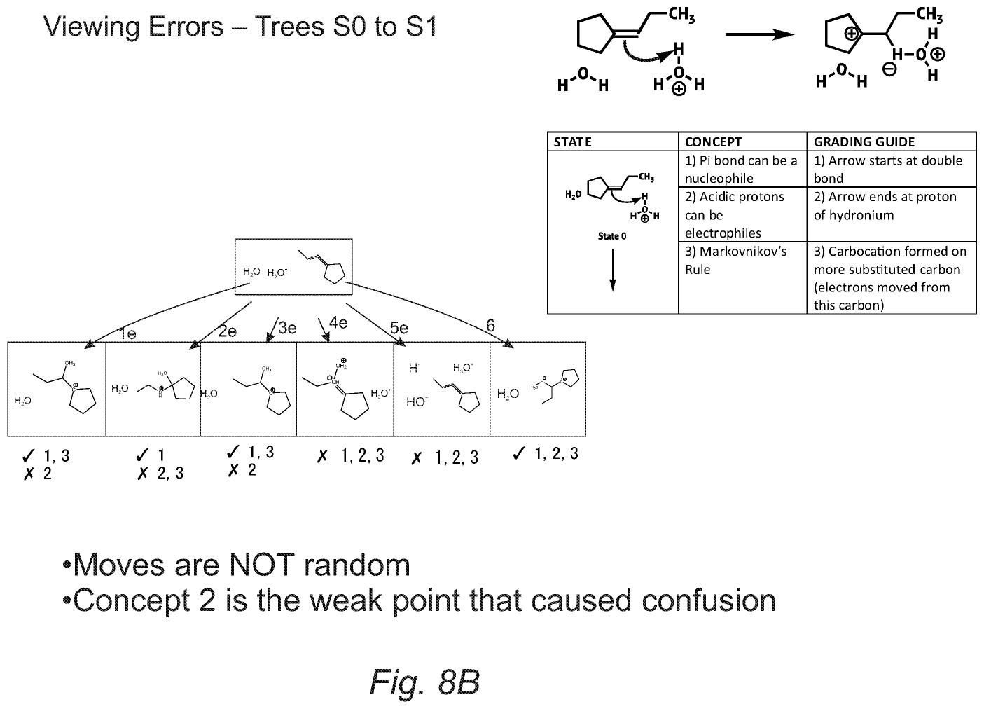

FIGS. 8A and 8B provide a schematic demonstrating a method for evaluating (e.g., scoring or grading) users' attempts in solving the mechanism puzzle. In this regard, a grading rubric is created is which individual steps in a mechanism problem are identified. For each step, relevant chemical concepts are identified and associated thereto. Examples of such concepts are found in Table 1. Other concepts relevant to chemistry are included in Table 2.

TABLE 2
Examples of chemical concepts

| | |
|---|---|
| 1. | Pi bond can be a nucleophile |
| 2. | Acidic protons can be can be |
| | electophiles |
| 3. | Markonvikov's Rule |
| 4. | Pi bond as a base |
| 5. | H₃O⁺ is an acid |
| 6. | O of water electron rich |
| 7. | Use O lone pair |
| 8. | O acts as Nucleophile |
| 9. | Carbocation is an electrophile |
| 10. | O of water electron rich |
| 11. | O acts as base |
| 12. | H of R₂OH⁺ is acidic (R is alkyl) |
| 13. | (C═C) Pi bond electron rich (LB) |
| 14. | Pi bond as a base |
| 15. | Anti-Markovnikov's rule |
| 16. | Electrons move away from proton |
| | and stay with acid |

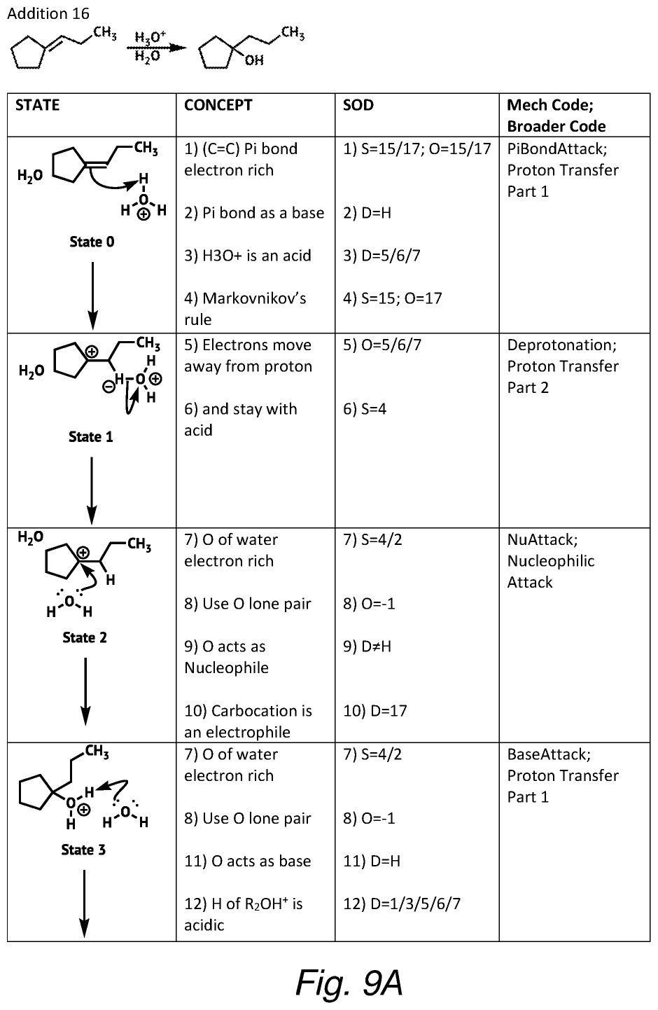

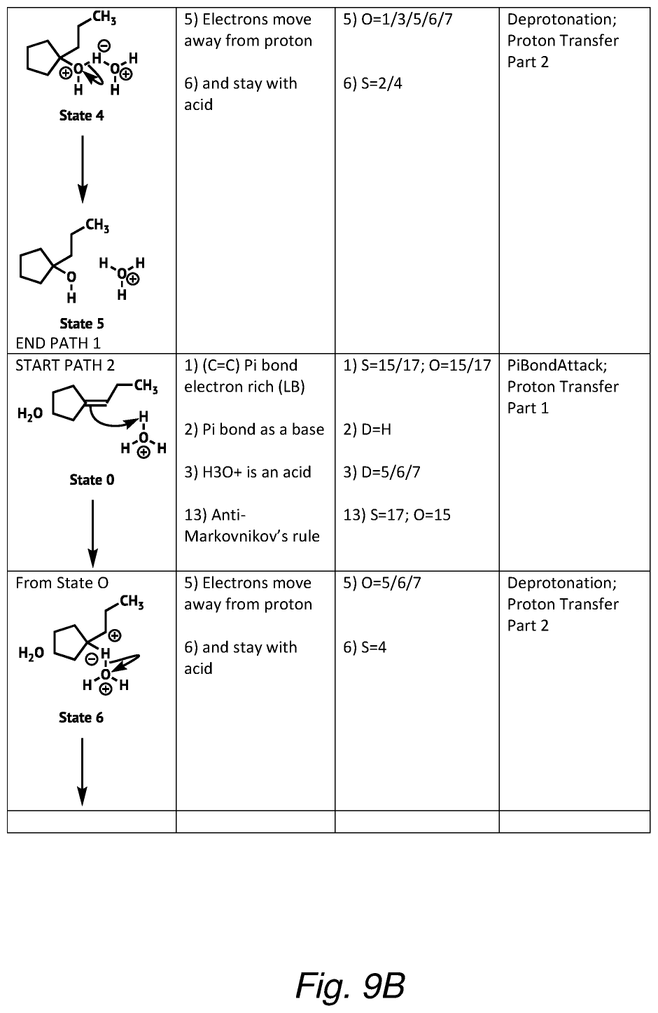

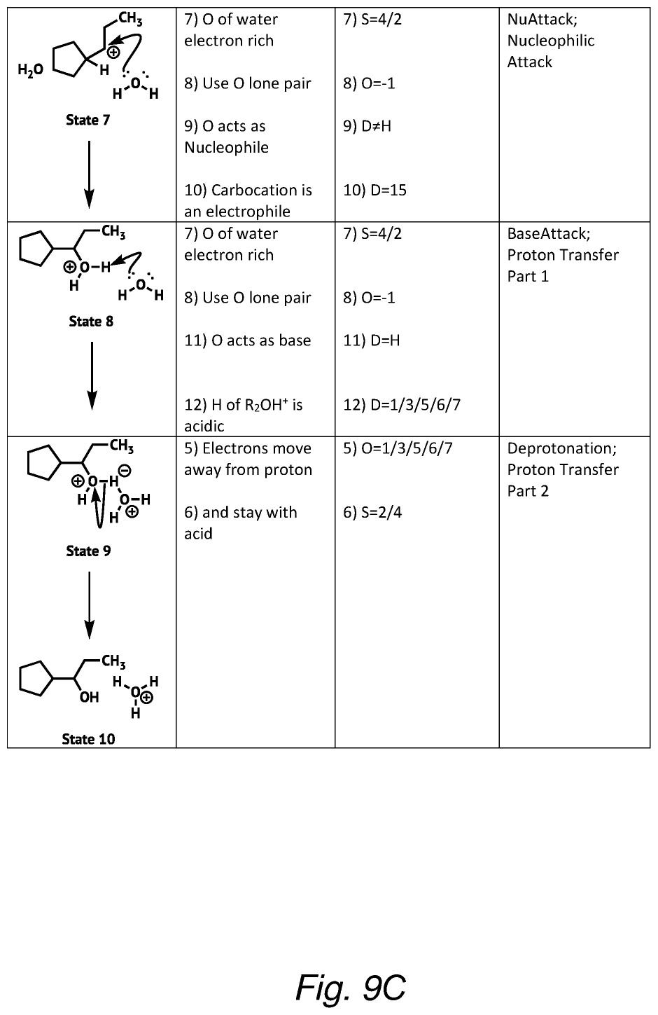

Once a list or database of chemical concepts is created, a grading rubric (e.g., a grading guide) can be applied by the tool to score a user's attempt a solving a problem as depicted in FIGS. 8A and 8B. FIGS. 9A, 9B, and 9C depict the application of a grading rubric to a multistep addition reaction mechanism. In these figures, “SOD” means source, origin, destination which designates from which atoms electrons come and go.

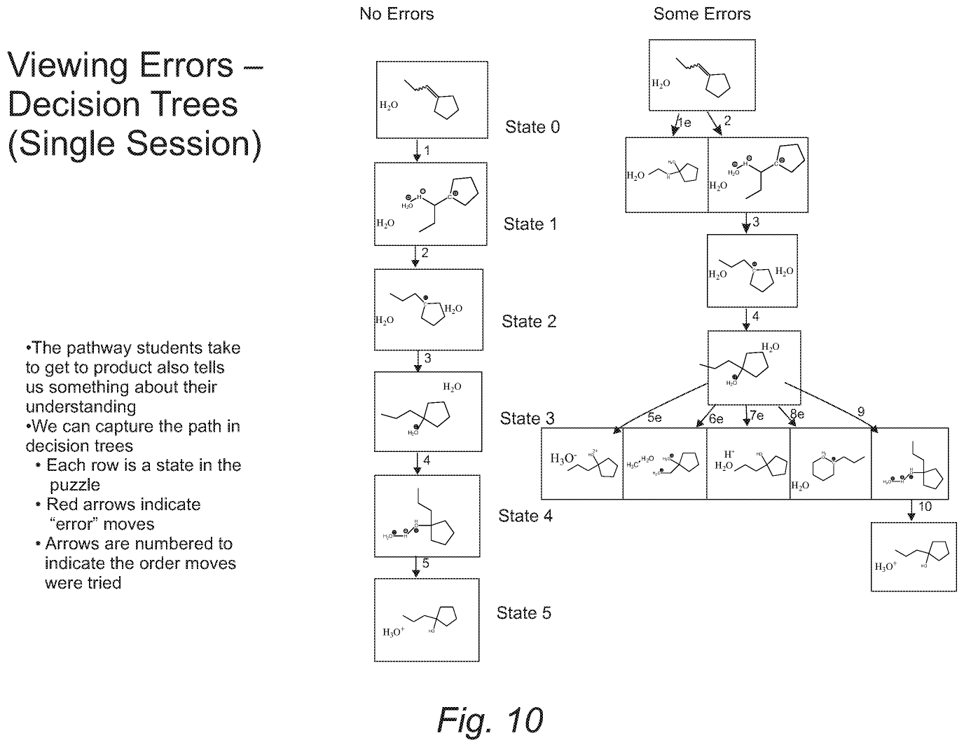

FIG. 10 provides a schematic showing that errors that users make can aid in identifying concepts that present difficulties. A useful feature of the present invention is that moves from a plurality of users can be collected on a central computer device (e.g., a server or a teacher's computer) and classified as correct or incorrect. In a refinement, individual moves can be further parsed into chemical concepts as set forth above. The users' success or failure in identifying such chemical concepts can also be stored on the central computer device.

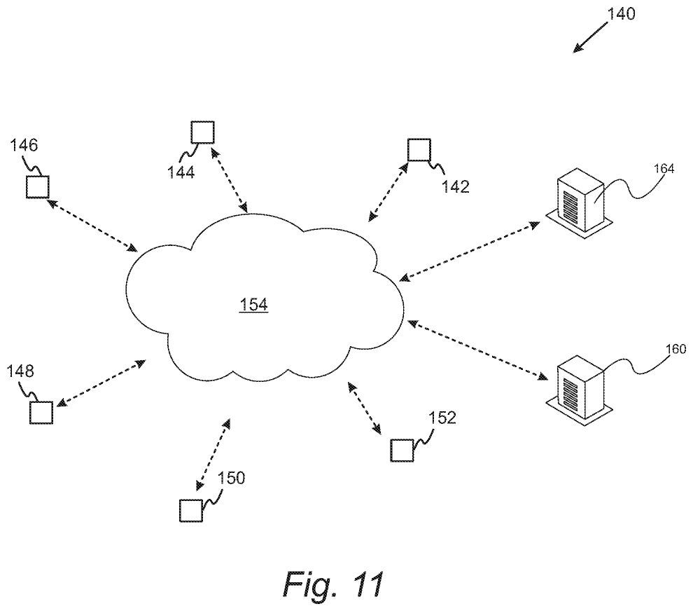

With reference to FIG. 11, a schematic of a networked chemical mechanism evaluation and data collection system is provided. Networked mechanism teaching and data collection system 140 includes one or more user computer devices 142-152 (e.g., 1 or 2 to 10,000 or more) communicating over network 154. The functionality of the user computer devices is set forth above. Network 154 can be a wired and/or wired network. Typically, network 154 will operate over the internet. Networked mechanism teaching and data collection system 140 includes one or more monitoring tools 160 with the functionality set forth above. In a refinement, monitoring tool 160 store tracking data on database 162 either implemented on monitoring tool 160 or a centralized server 164. Also depicted in FIG. 10 is authoring computer tool 166 also described above.

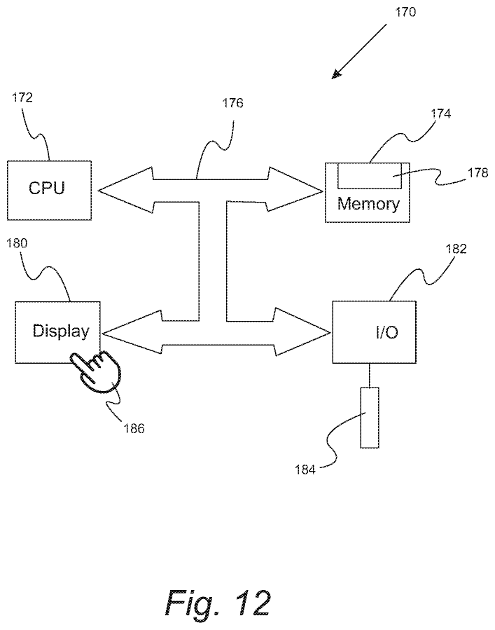

The methods set forth above involve both an authoring computer device and a user computer device. In general, both computer devices are computer processor-based electronic devices and will be referred to as computer device 10. With reference to FIG. 12, computer device 170 includes computer processor 172 that executes the instructions for authoring the chemical mechanism problem or solving it. It should be appreciated that virtually any type of computer processor may be used, including microprocessors, multi-core processors, and the like. The instructions for the method typically are stored in computer memory 174 and accessed by computer processor 172 via connection system 176. In a variation, connection system 176 is and/or includes a data bus. In a refinement, computer memory 174 includes a computer-readable medium 178 which can be any non-transitory (e.g., tangible) medium that participates in providing data that may be read by a computer. Specific examples for computer memory 174 include, but are not limited to, random access memory (RAM), read-only memory (ROM), hard drives, optical drives, removable media (e.g., compact disks (CDs), DVD, flash drives, memory cards, etc.), and the like, and combinations thereof. In another refinement, computer processor 12 receives instructions from computer memory 174 and executes these instructions, thereby performing one or more processes, including one or more of the processes described herein. Computer-executable instructions may be compiled or interpreted from computer programs created using a variety of programming languages and/or technologies including, without limitation, and either alone or in combination, Java, C, C++, C#, Fortran, Pascal, Visual Basic, Java Script, Perl, PL/SQL, etc. Display 180 is also in communication with computer processor 172 via connection system 176. Computer device 170 also includes various in/out ports 182 through which data from a pointing device 184 may be accessed by computer processor 172. Examples for the computer device include, but are not limited to, desktop computers, laptops smartphones, tablets, or tablet computers. Specifically, the methods can be implemented by iPad, iPod, and other tablets. Examples of pointing devices include a mouse, touch screen, stylus, trackball, joystick or touchpad. In a particularly useful variation, the pointing device is incorporated into display 178 as a touch screen by which user 186 interacts with a finger. In a variation, a non-transitory storage medium or media as set forth above has encoded thereon instructions for the steps executed by the user tool and/or the authoring tool and/or the monitoring tool.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method comprising:
authoring a chemical mechanism problem to be solved by a user with an authoring tool, the chemical mechanism problem presenting the user with chemical renderings of starting chemical compounds to be rearranged in a predetermined series of steps to form a predetermined final chemical compound, the authoring tool being implemented by an authoring computer device having a processor and a display, the chemical renderings of starting chemical compounds to be displayed on a user computer device;
receiving a series of inputs from a user tool on the user computer device for moving atoms and or bonds in the chemical rendering of starting chemical compounds to reproduce a chemical mechanism;
characterizing and storing moves made by the user; and
collecting the moves made by the user on a centralized computer device.

2. The method of claim 1 wherein a first listing of atoms is presented by the authoring tool to be used in authoring the chemical mechanism problem, the author selecting an atom from the first listing of atoms and placing the selected atom on a design region.

3. The method of claim 1 wherein implied hydrogen atoms are displayed by a letter “H” orbiting a carbon atom represented by a letter “C” on either the authoring computer device or the user computer device, the number of letters “H” representing the number of implied hydrogen atoms bonded to the carbon atom.

4. The method of claim 3 wherein implied hydrogen atoms orbit on displayed circles surrounding the letter “C.”

5. The method of claim 3 wherein the size of the letter “H” is smaller than the size of the letter “C.”

6. The method of claim 3 wherein upon actuation, each letter “H” is replaced by a rendering of a hydrogen atom bonded to carbon.

7. The method of claim 1 further comprising identifying frequent errors made by individual users.

8. The method of claim 1 further comprising identifying frequent errors made by a plurality of uses.

9. The method of claim 1 further comprising identifying patterns of steps made by individual users.

10. The method of claim 1 further comprising identifying patterns of steps made by a plurality of users.

11. The method of claim 1 wherein potential errors that the user may commit are identified during authoring of the chemical mechanism problem.

12. The method of claim 11 wherein a message to be displayed if the user commits an identified potential error.

13. A networked chemical mechanism evaluation and data collection system comprising:
a plurality of user computer devices, each user computer device executing a user tool that can receive a chemical mechanism problem, the chemical mechanism problem presenting a user with chemical renderings of starting chemical compounds to be rearranged in a predetermined series of steps to form a predetermined final chemical compound, the user tool receiving a series of inputs for moving atoms and or bonds in the chemical rendering of starting chemical compounds to reproduce a chemical mechanism; and
a monitoring tool that tracks the series of inputs for each user computer device.

14. The networked chemical mechanism evaluation and data collection system of claim 13 further comprising an authoring tool with which the chemical mechanism problem to be solved by the user is authored, the authoring tool being implemented by an authoring computer device having a processor and a display.

15. The networked chemical mechanism evaluation and data collection system of claim 13 wherein the monitoring tool characterizes and stores moves made by the user.

16. The networked chemical mechanism evaluation and data collection system of claim 15 wherein collected moves are stored in a database on a centralized computer device.

17. The networked chemical mechanism evaluation and data collection system of claim 16 wherein the monitoring tool identifies frequent errors made by a plurality of users.

18. The networked chemical mechanism evaluation and data collection system of claim 16 wherein the monitoring tool identifies patterns of steps made by a plurality of users.

19. The networked chemical mechanism evaluation and data collection system of claim 18 wherein an expert system is used to identify the patterns of steps made by the plurality of users.

20. The networked chemical mechanism evaluation and data collection system of claim 18 wherein a trained neural network is used to identify the patterns of steps made by the plurality of users.
